(19) United States
(12) Patent Application Publication
(10) Pub. No.: US 2005/0149176 A1
(43) Pub. Date: Jul. 7, 2005

(54) Selectively light curable support members for medical devices

(75) Inventors: James Heggestuen, Stillwater, MN (US); Thomas J. Holman, Princeton, MN (US); Jan Weber, Maple Grove, MN (US)

Correspondence Address: CROMPTON, SEAGER & TUFTE, LLC, 1221 NICOLLET AVENUE, SUITE 800, MINNEAPOLIS, MN, 55403-2420, US

(73) Assignee: SciMed Life Systems, Inc.

(21) Appl. No.: 10/750,579

(22) Filed: Dec. 29, 2003

(51) Int. Cl. A61F 2/06

(52) U.S. Cl. 623/1.46

(57) ABSTRACT

Methods of forming support layers for use in catheters using having a support layer included, and stents incorporating coatings of photosensitive polymerizable resins and stents including fibers coated with photosensitive polymerizable resins. A fiber is coated with a PPC resin and incorporated into a support structure for a catheter. Portions of the PPC are polymerized by exposure to light of a desired wavelength, causing increased rigidity and strength to the polymerized portions. As the PPC is polymerized, the fibers coated by the PPC resins become stronger and change the flexibility of devices incorporating such fibers. Additional embodiments include stents incorporating PPC coatings and methods of using such stents, including polymerizing a PPC coating after inserting a self-expanding or balloon-expandable stent.

### FIELD OF THE INVENTION

The present invention relates generally to support members used to provide improved properties to catheters, stents. More particularly, the present invention relates to support structure designs wherein the stiffness and/or shape of the support structure can be altered due to selective curing by light.

### BACKGROUND OF THE INVENTION

Many medical procedures include the insertion of a catheter into a lumen of a living body. Catheters are commonly used in procedures in the vascular system such as angiography, angioplasty, and other diagnostic or interventional procedures. In many of these procedures, the catheter must travel a tortuous path in order to reach the point of treatment. In order to aid in this travel through a body lumen, it is often desirable to have variable stiffness along the shaft of the catheter. With balloon catheters, various material transitions may be used to effect variable stiffness. Alternatively, a stiffening support structure such as a stainless steel braid may be included in the catheter shaft, and the braid may have varying PIC or other properties to modify stiffness along the axial length of the catheter shaft.

Guide catheters are often used to protect and guide a balloon catheter to a location near a treatment site. Typically, guide catheters will use a triple layer construction with a lubricious inner layer, an intermediate support layer, and a relatively soft outer layer. Often, a guide catheter may be given a preformed shape. For example, the distal portion of a guide catheter may have a hooked shape allowing it to hook into the left ascending aorta of a patient. Because of individual physical characteristics, different patients may require the stiffness changes to be at different points along the length of the catheter or may require variations in the shape of the catheter shaft. One way to modify catheter properties is to provide a thermoplastic catheter shaft that can be heated and shaped with hot water or when exposed to another heat source. The shaping of the catheter can then be performed by the clinician. However, this thermal process can also affect other properties of the thermoplastic (for example, brittleness or tensile strength) or the shape of the shaft or of a lumen therethrough, and the procedure can be imprecise.

The use of stents to prevent restenosis after an angioplasty treatment has become common practice. A stent is placed in collapsed form over a balloon of an angioplasty catheter. When the balloon is expanded, the stent expands to the inflated outer profile of the balloon, which is most likely not similar to the most preferred anatomical shape of the vessel in which it is place. For example, strong curvatures or taperings. Further, the steps of collapsing and placing a stent over a balloon can be labor intensive and difficult to perfect. Alternatively, a self-expanding stent may be collapsed and held within a retaining structure such as a delivery catheter. When delivered to a desired location, the self-expanding stent is expelled from the retaining structure and expands from its compressed state. Self-expanding stents have a tendency, however, to lack sufficient strength to maintain their expanded shape. For many stents, a metallic structure is used. However, a metallic stent is typically not conducive to the use of MRI diagnostic techniques that are used for a number of reasons. Meanwhile, nonmetallic stents often lack desired properties (i.e., strength) that can make them usable for this purpose.

Another limitation with respect to stent technology is that existing stents are made with materials that are relatively stiff. For many applications, such as peripheral vasculature aneurysm treatments, reduced profile during insertion is quite important. However, as the profile of the collapsed stent during insertion is reduced, the portion of the catheter section where the stent is disposed becomes stiffer. This makes placement of the stent in a desired location difficult.

### SUMMARY OF THE INVENTION

One embodiment of the invention includes a catheter shaft section comprising a support member. The support member may be formed using any suitable structure, i.e., tubes, braided, coiled, or woven designs, or other structures that use one or more strands to make a tubular member. At least one strand used in making the support member comprises a fiber coated with a resin comprising a photosensitive polymerizable composition (PPC). To facilitate coating with the resin, the fiber may be treated by a plasma treatment or other treatment to improve adhesion with the PPC. A single fiber may comprise a group of filaments. The filaments may be individually short in length, but part of a long or endless fiber. The plasma treatment, in this instance, will facilitate the coating of each of the filaments and to fill the space between filaments to bind together and form a fiber.

Another embodiment includes a guide catheter incorporating a support member as just described. A further embodiment includes a method comprising the step of providing a guide catheter including a support structure comprising a number of fibers and a PPC resin. The method includes shaping the guide catheter by the steps of holding the guide catheter in a desired shape and exposing portions of the guide catheter to light that causes at least partial polymerization of the resin. The supporting material of the catheter is preferably chosen to allow sufficient light access, transparency, to the fibers. One possible polymer is a clear polyamide to for a suitable matrix.

Another illustrative embodiment includes a balloon catheter. The balloon catheter may include portions that have a support structure in the form of a braid or other tubular member, wherein the support structure includes a PPC resin. The support structure has a varying stiffness over its length because certain portions of the support structure include more polymerized PPC resin than other portions. Another embodiment includes a method for using such a balloon catheter including the step of exposing at least a portion of the catheter to light to at least partially polymerize the PPC resin.

Yet a further illustrative embodiment includes a support structure for an elongate lo medical device such as a catheter. The support structure includes a number of fibers formed into a braided, coiled, woven, or other tubular member. At least some of the fibers are coated in certain locations with a PPC resin. The fibers may be pre-treated to encourage adhesion to the PPC resin. Additional embodiments include methods for making and using, as well as devices incorporating, such a support structure. In some such embodiments, the amount, type, or other characteristics of PPC resin provided at different locations along the length of the support structure may vary.

Another illustrative embodiment includes a stent that can be used to support a bodily lumen such as a blood vessel. The stent includes portions comprising fibers coated by a PPC resin. The PPC resin coated fibers may be stiffened once the stent is in place, or may be stiffened prior to insertion to a body lumen.

An illustrative method embodiment includes providing a stent having portions comprising fibers coated by a PPC resin. The stent may be collapsed onto a balloon or other expandable catheter by folding at least some of the PPC resin coated fibers. The method may further include advancing the stent to a desired location in a bodily lumen and expanding the stent at the desired location. The stent may then be exposed to light to cause at least some of the PPC resin to polymerize, causing the stent to stiffen in its expanded state. Allowing the vessel time to reshape the stent, prior to stiffening, to a more preferred shape helps overcome the issue of shape mismatch between the expanded balloon shape and vessel anatomy. This is a definite advantage over stent structures unable to be stiffened in-vivo.

### BRIEF DESCRIPTION OF THE DRAWINGS

FIGS. 1A-1B are front and cross-sectional views, respectively, of a single fiber strand including a PPC resin coating;

FIG. 2A is a front view of a braided set of fibers;

FIG. 2B is a front view of a braided set of coated fibers;

FIGS. 2C-2D are front and cross-sectional views, respectively, of a braided multi-fiber strand including a PPC resin coating;

FIG. 3 is a front view of a braided support structure incorporating a strand having a PPC resin coating;

FIG. 4A is a side view of a generally straight catheter;

FIG. 4B is a cross-sectional view taken along line B-B in FIG. 4A;

FIG. 4C is a side view of the distal end of the catheter of FIG. 4A after being curved and cured;

FIG. 4D is a top view of an illustrative catheter curve curing table;

FIG. 5 is a cross-section of a catheter shaft incorporating a multi-fiber strand coated with PPC resin in a support structure;

FIGS. 6A-6C illustrate in front views a method of cutting a reinforcing member while also capturing loose filaments at the cut end;

FIG. 7A illustrates an exemplary stent design;

FIG. 7B illustrates the end of a stent wrapped beneath a braided fiber strand having a PPC coating;

FIG. 8A illustrates a stent design incorporating PPC coated strands; and

FIG. 8B illustrates an alternative stent design incorporating PPC coated strands.

### DETAILED DESCRIPTION OF THE INVENTION

The following detailed description should be read with reference to the drawings. The drawings, which are not necessarily to scale, depict illustrative embodiments and are not intended to limit the scope of the invention. As used herein, the term “light” includes radiation of any wavelength and is not limited to visible, infrared, or ultraviolet wavelengths.

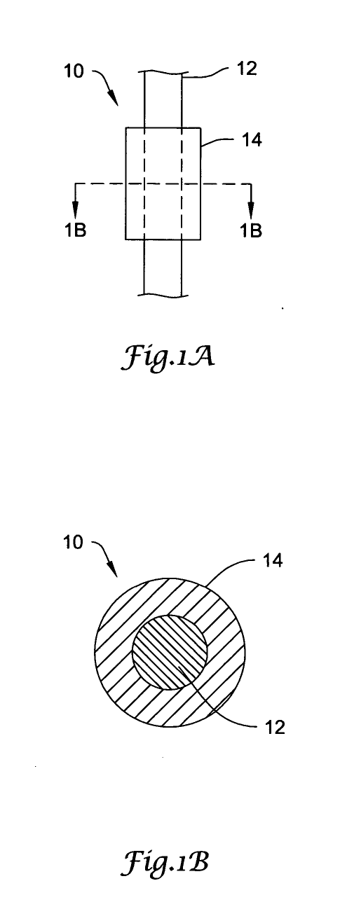

FIGS. 1A-1B illustrate a strand for use in catheter support members and stents, with FIG. 1B being a cross section along line B-B in FIG. 1A. The strand 10 includes a fiber 12 and a coating 14. The fiber 12 may be metal or non-metallic, and preferably is a polymer fiber. One suitable polymer is a high strength polyethylene fiber sold under the brand Spectra™ by Honeywell®, which is used in Spectra Shield® protective (i.e., bulletproof) materials. Ceramic fibers from 3M or Nextell could also be utilized. Preferably, the coating 14 is a photosensitive polymerizable composition (PPC) which, when exposed to a certain wavelength (or band of wavelengths) of light/radiation, undergoes a chemical change wherein the resin begins to form polymer chains. This polymerization preferably causes the strand 10 to become less pliable and more stiff. The coating 14 may include a ceramic or composite resin having, for example, zirconium or the like. Some available PPC resins include Renamel®, marketed by Cosmedent®, a Deltamed GmbH company, or Supreme™ made by 3M®. In a preferred embodiment, the fiber 12 is pre-treated in a cold gas plasma to improve adhesion of the coating 14 to the fiber 12. This treatment creates oxygen “hand-holds” on the fiber 12 to which the coating 14 can chemically bond. It is believed the treatment creates chemical groups like carboxylic acid or hydroxylic acid, which foremost improves wetability of the fiber and could provide chemical bonds. Other processes could also be used, such as hot plasma, UV top layer ablation or chemical etching.

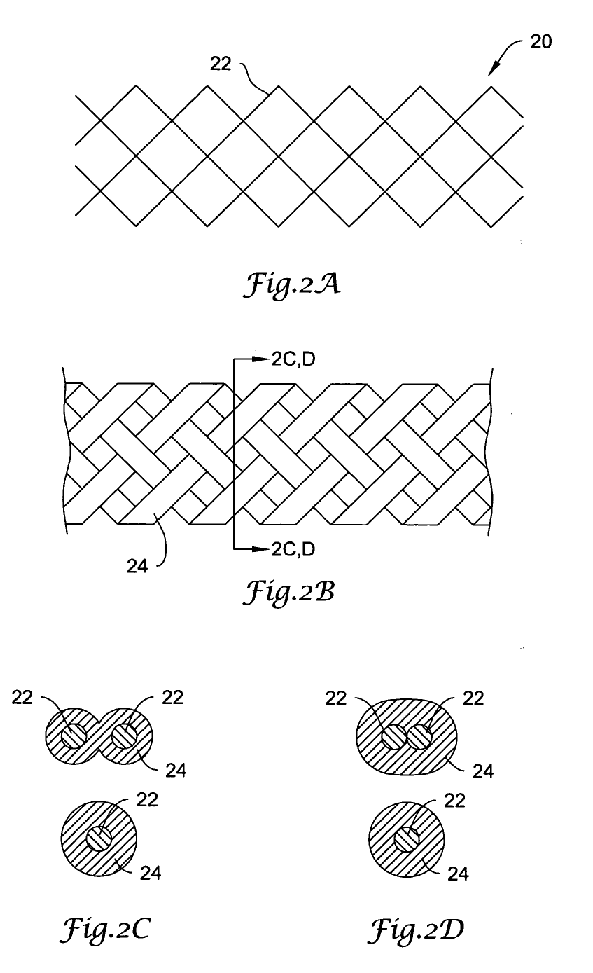

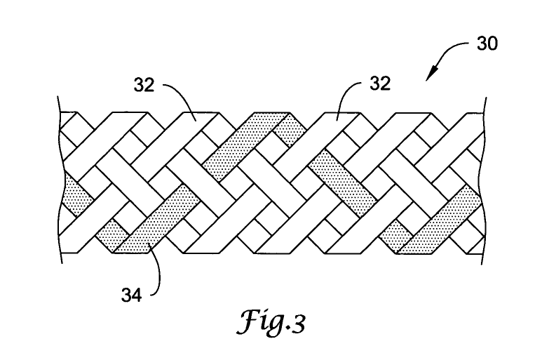

FIGS. 2A-2D illustrate a ribbon of coated fibers usable as a strand for use in catheter support members and stents. While FIGS. 1A-1B illustrate a single strand having one fiber and a coating thereon, an alternative device and method for forming such a coated element is shown by FIGS. 2A-2C. In particular, as shown in FIG. 2A, a number of fibers 22 may be manipulated into a mesh, braid or weave 20. Once so manipulated, the fibers 22 are then coated or saturated with a coating 24 that may be similar to coating 14 of FIG. 1, as shown in FIG. 2B and in cross section in FIGS. 2C-2D. FIG. 2C illustrates a cross section for fibers that are first coated and then braided before curing, and FIG. 2D shows fibers that are braided before coating. In particular, the coating 24 preferably comprises a PPC as part of the resin in the coating. While a flat ribbon is shown in FIGS. 2A-2D, round, multi-layer, or other structures may also be formed of the fibers 22.

As used herein, the term “strand” includes both individual coated fibers as shown in FIGS. 1A-1B, or may include a structure comprised of a number of fibers as shown in FIGS. 2A-2C. Where a multi-element or mono-element strand is preferable in the following illustrative embodiments, it will be noted. In general, embodiments using multi- and mono-element strands are contemplated as within the scope of the present invention. A single fiber can comprise a group of filaments. The filaments may be individually short in length, but together form a continuous fiber. The plasma treatment coats each short filament and fills the space between filaments, thus connecting the filaments into a single fiber.

The PPC resins and coatings used in the strands of FIGS. 1A-1B and 2A-2C preferably include either a photoiniferter or a photoinitiator. A photoinitiator causes the polymerization of the resin to begin once exposed to the activating wavelength of light, but does not halt the reaction when the irradiation of the activating wavelength stops. Preferably, however, a photoiniferter is used. The background of photiniferters as well as their use in dental applications is disclosed in U.S. Pat. No. 5,449,703, the disclosure of which is hereby incorporated by reference. In short, a photoiniferter causes polymerization of the resin to occur only while exposed to an activating wavelength of light, and the reaction stops when irradiation by the activating wavelength ends. The reaction may be later restarted by additional exposure.

In one embodiment, a radiopaque filler material may be provided in at least portions of the coating. In such an embodiment, the use of a radiopaque filler material in portions of the coating may allow for incorporation of marker bands in the support structure of a stent or catheter. For example, in particular with catheters, the addition of radiopaque marker bands adds steps to the fabrication process. If the strands are coated by the use of a spray-on process, the material that is spray deposited may be varied along the length of a strand to create marker bands where desired. Variation of the spray material can be accomplished, for example, by simply controlling the blend of material fed to a spray nozzle. By incorporation of such marker bands in the support structure for a catheter that makes use of such strands, the process of fabricating a catheter can be simplified. Preferably, the PPC also includes a ceramic type of filler material such as Zirconium.

The PPC resin may also include any number of accelerants that speed the polymerization reaction, stabilizers, monomers chosen to affect the properties of the resulting polymer structure, and photosensitizers that may improve the ability of the PPC resin to absorb and respond to irradiation. The particular activating wavelength of the PPC can vary widely within the scope of the present invention. In several embodiments, easily shielded or avoided wavelengths are preferred. For example, some embodiments make use of an ultraviolet wavelength for the activating wavelength. This may allow easy preparation and handling during both fabrication and surgical procedures, as non-UV emitting lights and filters for use with UV emitting lights are available, such devices being known for use in microfabrication laboratories, for example.

Other wavelengths that do not attenuate quickly in flesh may also be used. This feature would eliminate insertion of an optical fiber into the patient's body to irradiate the PPC resin as a process step. By removing the need for an inserted optical fiber, the duration of a procedure may be shortened, and the time during which a catheter and other devices are disposed in the patient's body is reduced. Further, the devices used for stent insertion may be simplified by the omission of an extra lumen for an optical fiber or, alternatively, by removing the need to incorporate an optical fiber in a catheter shaft.

The following several figures illustrate the inclusion of one or more strands including PPC resin coated fiber(s) in a number of medical devices. The particular structures shown are merely illustrative, enabling one of skill in the art to grasp how such strands and fibers may be incorporated into a number of instruments.

FIG. 3 is a front view of a braided support structure incorporating a strand having a PPC resin coating. It should be understood that the braided support structure 30 typically takes the form of a tubular member, but is much easier to show in a single dimensional view as shown in FIG. 3. A number of strands 32, 34 are illustrated. At least one strand 34 comprises a fiber as shown in FIGS. 1A-1B or a number of fibers as shown in FIGS. 2B-2D, coated with a PPC resin.

The support structure 30 may be formed by any of a number of known techniques for braiding, for example, by winding the strands 32, 34 onto a mandrel such as a metallic tube. The support structure 30 may then be relaxed, removed from the mandrel, and used in known methods for incorporating a tubular support structure in a catheter or the like. Alternatively, the support structure 30 may be wound onto a tubular polymeric member such as a PTFE tube, for example. After braiding/winding is completed, another polymer layer can be provided over the top of the braid, for example, by extrusion or the placement of heat shrink tubing. Alternatively, a layer including the light curable material can remain exposed and form the inner or outer layer of the device.

A wide variety of other forms of support structure 30 are also contemplated. For example, a helical coil, dual helical coils, coils wound in opposing directions, knit, crochet, or any configuration may be used. If desired, partial curing of portions of the support structure 30 may be performed before removal from a mandrel or incorporation into a catheter. For example, if only PPC coated highly flexible fibers are used, the support structure 30 may be difficult to handle until it is partially stiffened by curing the PPC coating on the polyethylene fibers. The use of a small beam laser or masking techniques enable selective irradiation of portions of the support structure 30, which can allow partial curing such that the structure remains flexible, yet is easily handled.

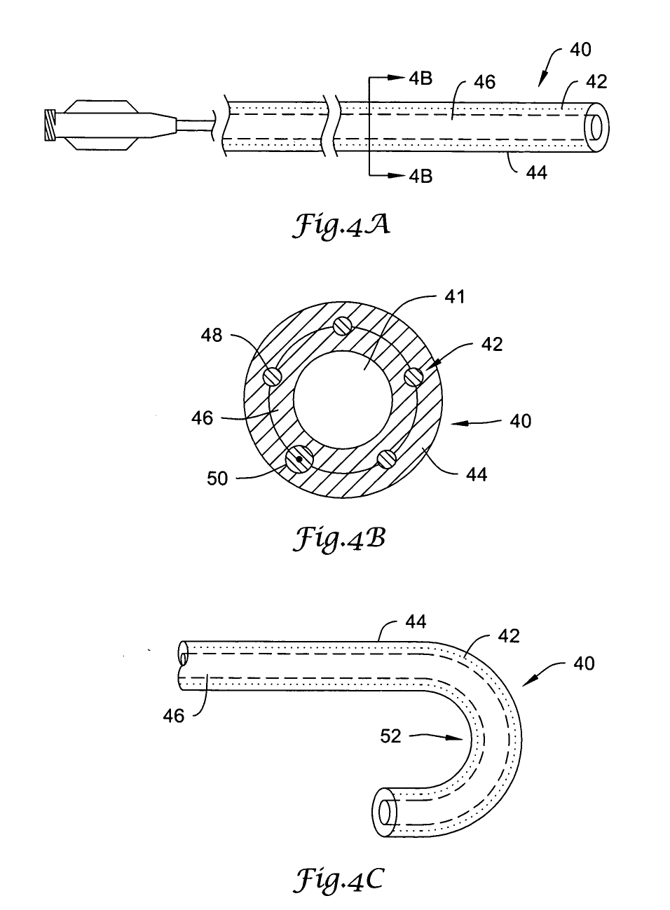

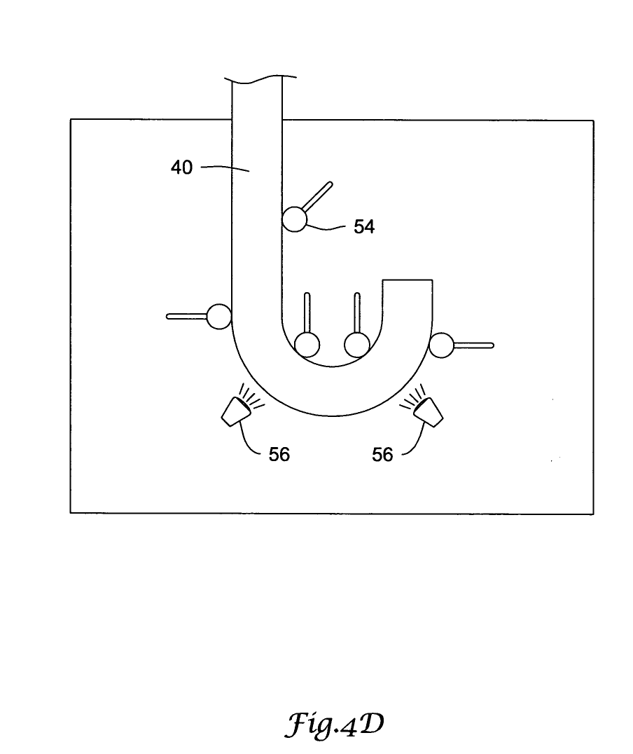

FIG. 4A is a side view of a generally straight catheter. The catheter 40 includes a reinforcing member 42 between an outer polymer layer 44 and an inner polymer layer 46. While the catheter 40 is illustrated as a guide catheter having a generally open distal end, the catheter 40 may take on any number of forms including, but not limited to, a balloon catheter, a cannula or an angiography catheter.

FIG. 4B is a cross-sectional view taken along line B-B in FIG. 4A. The catheter 40 defines a lumen 41, though in other embodiments, multiple lumens may be defined inside the inner polymer layer 46. The reinforcing member 42 includes a number of strands 48, 50. While some strands 48 may be ordinary metallic or non-metallic reinforcing strands, at least one strand 50 is a fiber including a PPC coating. In some embodiments, all of the strands 48, 50 may be fibers having PPC coatings.

FIG. 4C is a side view of the distal end of the catheter of FIG. 4A after being curved and cured. For example, the catheter 40 may be constructed in any of a variety of shapes, including the straight shape shown in FIG. 4A. A clinician (i.e., a physician, nurse or technician) may put the catheter 40 into a desired predetermined shape such as the curve 52 shown in FIG. 4C. The catheter 40 can then be irradiated with an activating wavelength for the PPC coating. Once irradiated, the PPC coated fiber strands 50 (FIG. 4B) stiffen, causing the catheter 40 to retain the desired shape and curve 52.

Although the shaping and curving may be performed manually, one may also use a specially designed table or mold to create accurate curvature. One such table is shown in FIG. 4D. The table may include pegs 54 and at least one radiation source 56. The pegs 54 may be movable within the table using, for example, a number of receiving holes in the table, or slidable channels on the table. Markings on the table may indicate particular sizes. It is readily appreciated that more complicated curves may also be created.

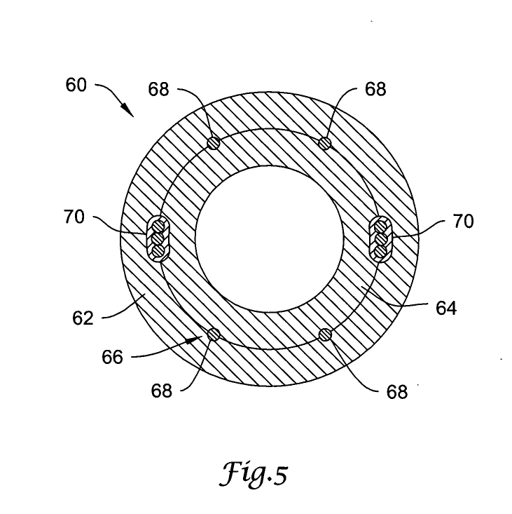

FIG. 5 is a cross-section of a catheter shaft incorporating a multi-fiber strand coated with PPC resin in a support structure. The shaft 60 includes an outer layer 62, an inner layer 64, and a support member 66 therebetween. The support member 66 includes a number of strands 68, 70. Some of the strands 68 may be ordinary strands such as metallic or non-metallic wires or ribbons, while at least one strand 70 is comprised of a number of fibers having a PPC coating. The fibrous strand 70 is illustrated as having several fibers wound or woven together with a PPC coating thereover.

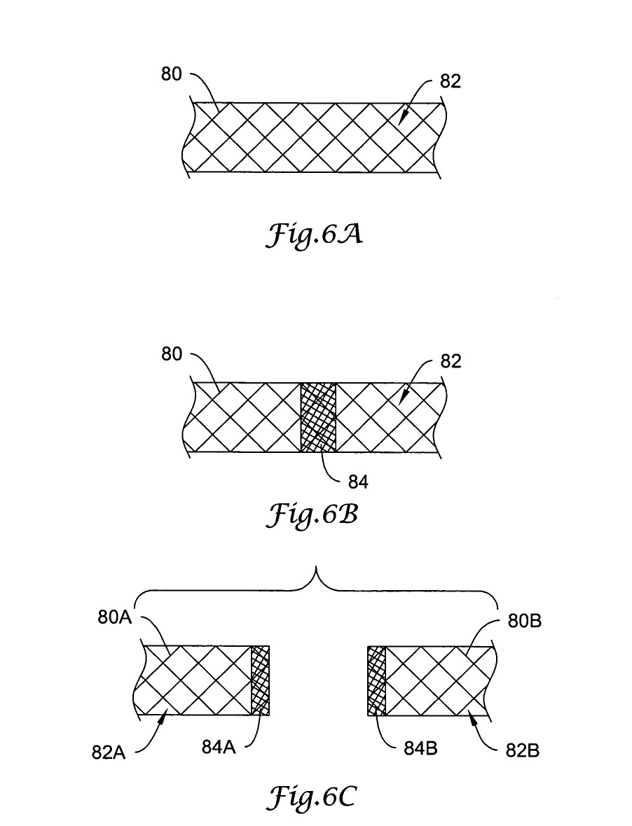

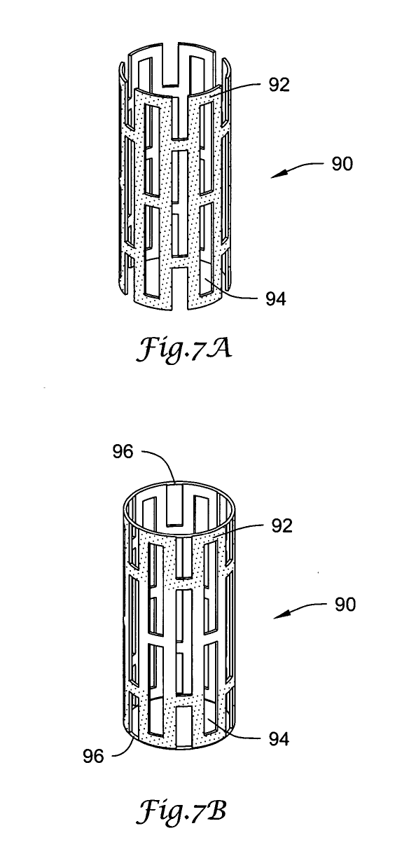

FIGS. 6A-6C illustrate in front views a method of cutting a reinforcing member while also capturing loose filaments at the cut end. Referring to FIG. 6A, the reinforcing member 80 is illustrated having a number of strands 82 that may be, for example, metallic or non-metallic ribbons or wires, or may also be fibers having a PPC coating. Referring to FIG. 6B, a PPC element 84 is placed at a chosen location over the strands 82. The PPC element may be, for example, a number of coated fibers wrapped around the reinforcing member 80, a number of fibers wrapped around the reinforcing member 80 and then coated, or simply a sprayed on coating of PPC material.

With the PPC element 84 placed, the reinforcing member 80 is then subjected to irradiation by an activating wavelength, causing the PPC to at least partially polymerize. Referring to FIG. 6C, the reinforcing member 80 is cut into a first reinforcing member 80A and a second reinforcing member 80B, with corresponding strands 82A, 82B and PPC elements 84A, 84B. One advantage of the illustrative process is that the reinforcing member 80 may be continually wound on a mandrel and fed out of the winding machine over the mandrel. If the mandrel comprises a number of sections that can be placed and/or removed, then winding can be continuous, with sections removed by the placement of the PPC elements 84 at chosen locations, with sections of the mandrel then being removed.

FIG. 7A illustrates one example of a stent. As shown, a stent 90 includes a strut-like structure 92 defining a number of gaps 94. Before, during and after placement, different properties of the stent structure are important. The stent must be pliable enough to collapse onto a deflated balloon and flexible enough to bend through tortuous anatomy. At the same time, the stent, when expanded, must have sufficient strength or rigidity to hold a vessel open. The present invention provides strength upon expansion by including a coating having a PPC polymer. This polymer can be selectively cured upon expansion of the stent. The PPC coating can include microfibers and filler material, such as a ceramic-like zirconium. The entire stent may be coated, or alternatively, only a portion of the stent as indicated in FIG. 7B. In FIG. 7B, only the end portions are coated to give structural support upon curing at these locations. The end portions 96 may be PPC coated with fibers or strands, and may be cured to cause at least partial polymerization. Again, because the end portions 96 can cured by a simple process step, properties of the stent prior to expansion are improved, plus a stronger expanded stent results upon light curing. Alternatively, the PPC can be embedded in graft material for a stent graft or a covering material for a covered stent.

Wallsten, in U.S. Pat. No. 4,655,771, provides an example of a self-expanding stent. One of the difficulties with self-expanding stents is the ability of the stent to fully expand and maintain its expanded shape. For example, self-expanding stents are often inserted to a body lumen by compressing the stent inside a tubular retainer, and when the tubular retainer is withdrawn, the stent elastically expands to a larger diameter. To enable compression without damage, the stent is typically made of relatively flexible materials that will not break under strain. Such materials, however, are often insufficiently rigid to hold their shape. The incorporation of curable strands in a self-expanding stent allows fabrication of a stent that is initially quite flexible but can be made rigid. The stent, once expanded, can be irradiated to stiffen the curable strands.

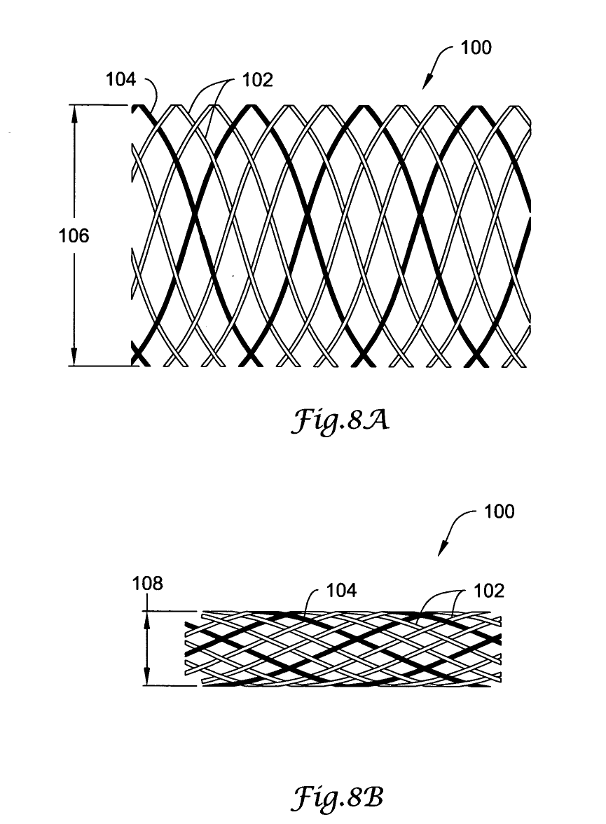

FIGS. 8A-8B are cutaway side views of a stent having a self-expanding structure, but including at least one PPC coated fiber. The stent 100 includes a number of strands 102, 104 in a helical structure, with at least some strands 104 comprised of PPC coated fibers. Preferably, some strands 104 are high molecular weight polyethylene strands that are cold plasma treated and then coated with a polymerizable coating that includes a photoiniferter. More preferably, the polymerizable coating also includes a ceramic material such as zirconium.

The stent 100 shown in FIG. 8A is shown in its expanded state, having a first diameter 106. Prior to insertion into a patient, the stent 100 is collapsed into a compressed state as shown in FIG. 8B, where the stent 100 has a second diameter 108 that is less than the first diameter 106. When compressed, at least some of the strands 102, 104 are out of their ordinary, stress-free state and exert a force radially outward. With a helical construction as shown, the stent 100 is more elongated in its radially compressed state than in the radially expanded state.

To perform an insertion, the stent 100 is first collapsed, and then placed inside a tubular restraint. The tubular restraint is typically an outer sheath that covers a catheter. To expand the stent 100, the tubular restraint is withdrawn with respect to the stent 100 by pushing the stent 100 distally of the distal end of the tubular restraint. If desired, a balloon catheter may be used as well, with the stent 100 disposed over the balloon such that the self-expanding forces of the stent are assisted by the pressure of the balloon.

Referring now to both FIGS. 8A and 8B, in one embodiment, some strands 102 comprise a springy metal or polymer. These strands 102 provide elastic or spring force that enables the self-expanding stent I 00 to self expand. One limit with springy materials is that, as time passes, when held in a single position, the spring tends to relax into the position it is held in, and fails to exert the same force. An advantage for the illustrative embodiment is that the curable strands 104 can be made rigid once in place to make the stent 100 resilient. For example, once expanded, a curing wavelength of light can be applied to the stent 100 causing the curable strands 104 to become rigid. By having a combination of “spring” strands 102 with a number of curable strands 104, the stent 100 retains the ability to self expand well and conform to lumen anatomy (i.e., in the vasculature, biliary tract, urinary tract, or elsewhere in the patient's body), while also being capable of becoming rigid when exposed to light of a particular wavelength.

Those skilled in the art will recognize that the present invention may be manifested in a variety of forms other than the specific embodiments described and contemplated herein. Accordingly, departures in form and detail may be made without departing from the scope and spirit of the present invention as described in the appended claims.

1. A method of constructing a support structure for a catheter comprising:
providing a first strand comprised of a fiber coated with a PPC resin; and
winding a number of strands including the first strand to form the support structure.

2. The method of claim 1, wherein said step of winding a number of strands includes forming a braid.

3. The method of claim 1, wherein said step of winding a number of strands includes forming a weave.

4. The method of claim 1, wherein said step of winding a number of strands includes forming a helical coil.

5. The method of claim 4, wherein the number of strands includes only the first strand.

6. The method of claim 1, further comprising only partially curing the PPC resin.

7. The method of claim 1, wherein the support structure has an axial length, the method further comprising:
curing the PPC resin to a first extent at a first location along the axial length of the support structure; and
curing the PPC resin to a second extend different from the first extent at a second location along the axial length of the support structure.

8. The method of claim 1, further comprising:
shaping the support structure to a predetermined shape; and
curing a portion of the PPC resin to cause the support structure to retain the predetermined shape.

9. The method of claim 8, wherein said shaping step is performed after the support structure has been incorporated into a catheter.

10. A method of providing a catheter with variable flexibility comprising:
providing the catheter with a reinforcing layer having at least one fiber coated with a PPC resin;
causing the PPC resin disposed on the fiber to polymerize to a first extent at a first location; and
causing the PPC resin disposed on the fiber to polymerize to a second extent at a second location;
wherein the first extent is different from the second extent.

11. The method of claim 10, wherein the first extent is greater than the second extent, and wherein the stiffness of the catheter is greater at the first location than the stiffness of the catheter at the second location.

12. The method of claim 10, wherein the step of causing the resin to at least partially polymerize includes exposing the wall of said catheter to radiation.

13. A support member for a catheter shaft section comprising:
a proximal end and a distal end;
a number of strands forming part of a tubular structure; and
an amount of a PPC resin coated on the strands near at least one of said proximal end or said distal end.

14. The support member of claim 13, wherein said PPC resin is at least partially polymerized.

15. The support member of claim 13, wherein said PPC resin encapsulates at least one of said proximal end or said distal end.

16. A stent for placement in a body lumen, the stent comprising:
a structure having a first end, and a second end; and
a PPC resin coated on at least one of said first end or said second end; wherein said PPC resin strengthens at least one of said first end or said second end upon expansion and curing.

17. The stent of claim 16, wherein said PPC resin is disposed about the encapsulated end by coating a fiber with PPC resin and wrapping the fiber around the encapsulated end.

18. The stent of claim 16, wherein said PPC resin is disposed about the encapsulated end by depositing said PPC resin on portions of the structure at the encapsulated end.

19. A catheter section comprising:
an inner polymeric layer;
an outer polymeric layer; and
a support structure between said inner layer and said outer layer, said support structure including at least one strand comprised of a fiber coated with a PPC resin.

20. The catheter section of claim 19, wherein said support structure is in the form of at least one helical coil.

21. The catheter section of claim 19, wherein said support structure is in the form of a braid.

22. A catheter comprising:
a first section having a first flexibility; and
a second section having a second flexibility that is greater than the first flexibility;
wherein both the first section and the second section include an inner layer, an outer layer, and a reinforcing layer including a fiber coated with a PPC resin therebetween;
wherein the first section includes a greater amount of polymerized PPC resin than the second section.

23. A method of forming a catheter comprising:
providing an inner layer;
providing an outer layer; and
providing a support member between said inner layer and said outer layer, said support member including at least one strand comprising a fiber coated with a PPC resin.

24. The method of claim 23, further comprising exposing a portion of said strand to light to cause at least partial polymerization of said PPC resin.

25. The method of claim 23, wherein said step of exposing said strand includes passing light through at least one of said inner layer or said outer layer.

26. A method of implanting a stent comprising:
providing a stent having at least one strand comprising a fiber coated with a PPC resin;
placing the stent over an expandable actuator including an electroactive polymer, the expandable actuator being disposed on an elongate medical device;
positioning the stent and the expandable actuator in a desired location in a body lumen;
actuating the expandable actuator by providing electrical energy; and
at least partially polymerizing the PPC resin by application of radiation to the stent.

27. The method of claim 26, wherein the fiber comprises a polyethylene fiber, and wherein the PPC resin includes a ceramic filler material.

28. The method of claim 27, wherein the step of providing a stent includes:
providing at least one polyethylene fiber;
cold plasma treating the polyethylene fiber to improve the adhesive characteristics of the polyethylene fiber; and
coating the polyethylene fiber with the PPC resin.

29. A method of implanting a self-expanding stent comprising:
providing a stent having at least a section coated with a PPC resin, the stent being elastically biased to a first diameter;
compressing the stent to a second diameter that is less than the first diameter against the elastic bias;
restraining the stent at a location near the distal end of a catheter shaft;
inserting the stent into the body of a patient by advancing the distal end of the catheter shaft into a body lumen of the patient;
releasing the stent at a desired location in the body lumen such that the stent expands using elastic restoring forces; and, after releasing the stent, at least partially polymerizing the PPC resin to stiffen the at least one fiber.

30. The method of claim 29, wherein the step of providing a stent uses at least one cold plasma treated polyethylene fiber coated with a polymerizable ceramic impregnated resin.

31. The method of claim 29, wherein the step of releasing the stent also includes inflating a balloon over which the stent was restrained to provide additional expansive force to the stent.

32. The method of claim 31, wherein the balloon is kept in an inflated state while the step of at least partially polymerizing the PPC resin is performed.

33. The method of claim 29, wherein the step of at least partially polymerizing the PPC resin is performed by irradiating portions of the stent with a desired wavelength.
